(19) United States
(12) Patent Application Publication
(10) Pub. No.: US 2022/0142820 A1
(43) Pub. Date: May 12, 2022

(54) ANTIMICROBIAL DRESSING, DRESSING COMPONENTS, AND METHODS

(71) Applicant: Systagenix Wound Management, Limited, Bracknell (GB)

(72) Inventors: Craig DELURY, North Yorkshire (GB); Sophie REGAN, Skipton (GB); Katie BOURDILLON, West Yorkshire (GB)

(21) Appl. No.: 17/433,424

(22) PCT Filed: Feb. 27, 2019
(86) PCT No.: PCT/IB2019/051591

§ 371 Date: Aug. 24, 2021

(51) Int. Cl. A61F 13/00; A61L 15/64; A61L 15/22; A61L 15/14

(52) U.S. Cl. CPC A61F 13/00012; A61L 15/64; A61L 15/225; A61F 13/00063; A61L 2300/104; A61F 13/00017; A61L 2300/404; A61L 2300/21; A61L 15/14

(57) ABSTRACT

A dressing that includes an absorbent, antimicrobial layer and a bioresorbable layer is disclosed herein. The absorbent, antimicrobial layer may be formed from nonwoven fibers. The bioresorbable layer may include an extracellular polymeric substance-active agent, such as citric acid. At least a portion of the nonwoven fibers may include carboxymethylcellulose fibers and alginate fibers.

### TECHNICAL FIELD

The claimed subject matter relates generally to therapy of a tissue site and, more particularly, but without limitation, to compositions and devices, including dressings and dressing components, for application to a tissue site such as a wound, and to methods related to the same.

### BACKGROUND

A wide variety of materials and devices, generally characterized as “dressings,” are generally known in the art for use in treating a wound or other disruption of tissue. Such wounds may be the result of trauma, surgery, or disease, and may affect skin or other tissues. In general, dressings may control bleeding, absorb wound exudate, ease pain, assist in debriding the wound, protect wound tissue from infection, or otherwise promote healing and protect the wound from further damage.

Some dressings may protect tissue from, or even assist in the treatment of, infections associated with wounds. Infections can retard wound healing and, if untreated, can result in tissue loss, systemic infections, septic shock and death. A variety of dressings containing antimicrobial agents are known in the art. Nevertheless, there remains a need for improved compositions having one or more characteristics such as improved antimicrobial efficacy, improved wound healing, improved absorption of blood and wound exudate, improved wound protection, reduced cost, and greater ease of use.

### SUMMARY

Compositions, for example, for use in a dressing component, dressings including such components, and methods related to the same are set forth in the appended claims. Illustrative embodiments are also provided to enable a person skilled in the art to make and use the claimed subject matter.

In an aspect, a dressing is provided that includes an absorbent, antimicrobial layer and a bioresorbable layer. In any embodiment herein, the absorbent layer may include absorbent fibers and antimicrobial fibers. In any embodiment herein, the bioresorbable layer may include an extracellular polymeric substance (EPS)-active agent. The bioresorbable layer may be configured to degrade at least a portion of a biofilm.

In a related aspect, a system for providing therapy to a tissue site is provided, the system includes a dressing of any embodiment disclosed herein. In a further related aspect, a method for providing therapy to a tissue site is provided where the method includes positioning a dressing of any embodiment disclosed herein adjacent to the tissue site.

### DRAWINGS

FIG. 1A is an illustrative representation of a cross-sectional, perspective view of an embodiment of a dressing of the present technology.

FIG. 1B is an illustrative representation of a cross-sectional, perspective view of an embodiment of a dressing of the present technology.

FIG. 1C is an illustrative representation of a cross-sectional, perspective view of an embodiment of a dressing of the present technology.

FIG. 1D is an illustrative representation of a cross-sectional, perspective view of an embodiment of a dressing of the present technology.

FIG. 2 is an illustrative representation of a cross-sectional, perspective view of the dressing of FIG. 1C, comprising one or more additional components.

FIG. 3 is a simplified schematic diagram of an exemplary embodiment of a negative pressure therapy system including a dressing of the present technology.

FIG. 4 is a graphical representation of the effect of dressings on 24 hour P. aeruginosa biofilms, according to the Examples.

It should be noted that the representative illustrations provided in the figures set forth herein is intended to illustrate the general features and/or characteristics of exemplary embodiments to aid in describing the present technology in full. The figures may not precisely reflect the characteristics of any given embodiment, and are not necessarily intended to define or limit the scope of the claimed subject matter. Further, the present technology may or may not include or incorporate therewith any one or more features of characteristics set provided in any one or more figures.

### DESCRIPTION

The following description provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

The present technology provides antimicrobial dressings, various layers thereof, and therapy systems including such dressings and/or layers, as well as methods including any embodiment disclosed herein of such dressings, various layers thereof, and/or therapy systems. Generally, and as will be disclosed herein, the dressings of the present technology may be configured to provide therapy to a tissue site.

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

As understood by one of ordinary skill in the art, “molecular weight” (also known as “relative molar mass”) is a dimensionless quantity but is converted to molar mass by multiplying by 1 gram/mole—for example, collagen with a weight-average molecular weight of 5,000 has a weight-average molar mass of 5,000 g/mol.

As used herein, the term “biofilm” refers to an association of microorganisms, e.g., single or multiple species, that can be encased or embedded in a matrix material, which may be self-produced by resident microorganisms. The biofilm may be present or adhere to living and/or non-living surfaces, e.g., tissue, a wound, medical implants, such as but not limited to orthopedic implants, dental implants, catheters, stents and so on. Exemplary microorganisms include, but are not limited to bacteria, e.g., Gram-negative bacteria, such as Pseudomonas aeruginosa, Gram-positive bacteria, such as Staphylococcus aureus and Streptococcus mutans, and fungi, such as yeasts, e.g., Candida albicans. The term “matrix material” is intended to encompass extracellular polymeric substances. Exemplary matrix materials include, but are not limited to polysaccharides, glycoproteins and/or nucleic acids. The term “biofilm” is further intended to include biological films that develop and persist at interfaces in aqueous environments. The language “biofilm development” or “biofilm formation” is intended to include the formation, growth, and modification of the bacterial colonies contained with biofilm structures, as well as the synthesis and maintenance of the exopolysaccharide of the biofilm structures. “Reducing” or “disrupting” a biofilm includes reducing the number of total viable microorganisms making up at least part of the biofilm, for example, as measured by total viable counts (TVC) of microorganisms (e.g., bacteria, yeast).

“Tissue site” as used herein refers to a wound, defect, or other treatment target located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, grafts, or a combination of any two or more thereof. The term “tissue site” may also refer to areas of any tissue that are not necessarily wounded or defective, but are instead areas in which it may be desirable to add or promote the growth of additional tissue.

As used herein, the term “effective amount” refers to a quantity sufficient to achieve a desired therapeutic effect, e.g., an amount which results in the decrease in a wound described herein or one or more signs or symptoms associated with a wound described herein. In the context of therapeutic applications, the amount of a composition administered to the subject will vary depending on the composition, the degree, type, and severity of the wound and on the characteristics of the individual. The compositions can also be administered in combination with one or more additional therapeutic compounds. In the methods described herein, the therapeutic compositions may be administered to a subject having one or more wounds.

As used herein, the terms “individual”, “patient”, or “subject” can be an individual organism, vertebrate, a mammal, or a human. In some embodiments, the individual, patient, or subject is a human.

“Treating” or “treatment” as used herein includes: (i) inhibiting a wound of a subject, i.e., arresting its development; (ii) relieving a wound of a subject, i.e., causing regression of the wound; (iii) slowing progression of a wound of a subject; and/or (iv) inhibiting, relieving, and/or slowing progression of one or more symptoms of a wound of a subject. Such treatment means that the symptoms associated with the wound are, e.g., alleviated, reduced, cured, or placed in a state of remission.

It is also to be appreciated that the various modes of treatment of wounds as described herein are intended to mean “substantial,” which includes total but also less than total treatment, and wherein some biologically or medically relevant result is achieved. The treatment may be a continuous prolonged treatment for a chronic wound or a single, or few time administrations for the treatment of an acute wound.

### Dressings of the Present Technology

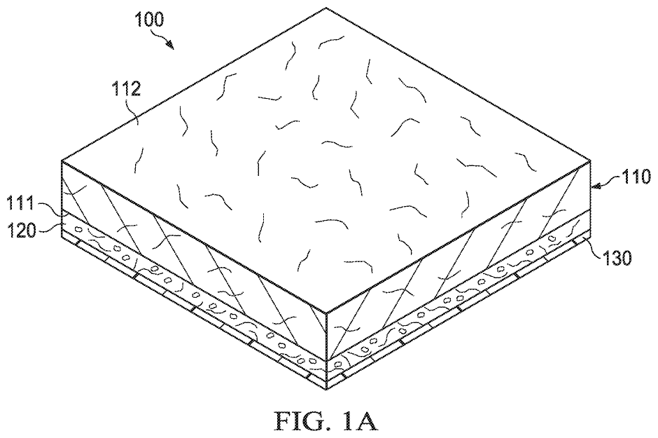

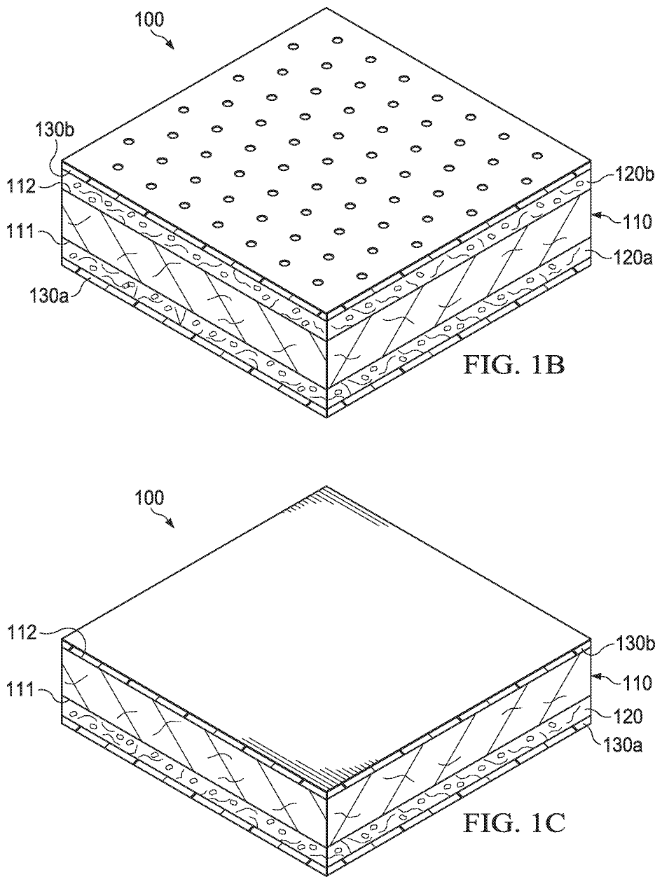

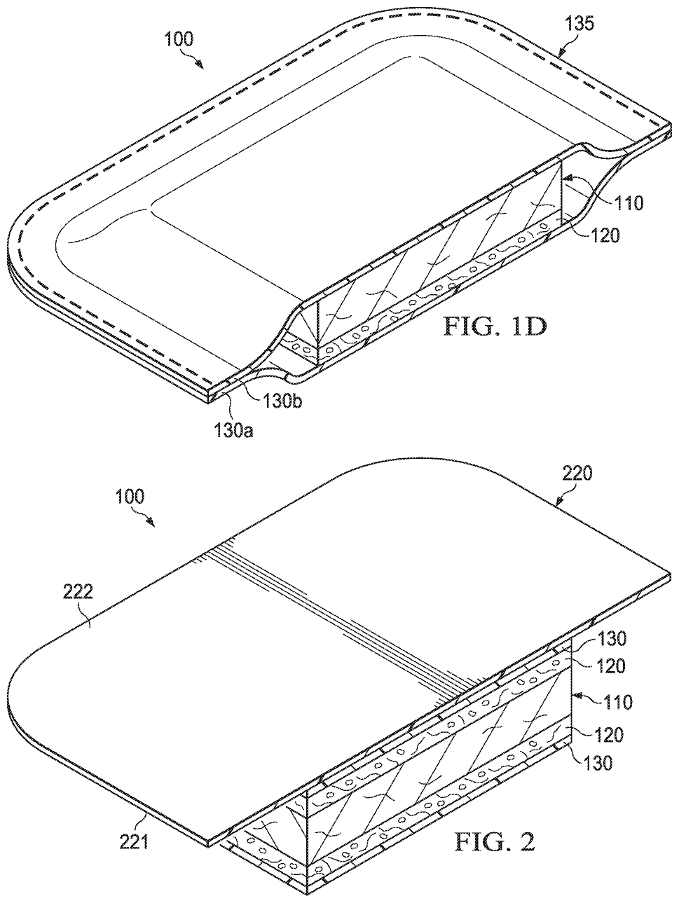

In an aspect, the present technology provides a dressing that includes an absorbent, antimicrobial layer and a bioresorbable layer. The bioresorbable layer includes an extracellular polymeric substance (EPS)-active agent. FIG. 1A provides an illustrative illustration of a dressing 100 that includes an absorbent, antimicrobial layer 110 and a bioresorbable layer 120. In any embodiment herein, the dressing may include layers, including but not limited to, an absorbent, antimicrobial layer, a bioresorbable layer, a non-adherent layer. For example, in any embodiment herein, the dressing may include an absorbent, antimicrobial layer, a bioresorbable layer configured to cover one or more surfaces of the absorbent, antimicrobial layer, and a non-adherent layer configured to cover a surface of the bioresorbable layer opposite the absorbent, antimicrobial layer. In any embodiment herein, the bioresorbable layer may be between the absorbent, antimicrobial layer and the non-adherent layer.

### Absorbent, Antimicrobial Layer

In any embodiment herein, the absorbent, antimicrobial layer may be a sheet having a generally planar configuration, including two generally planar surfaces opposite each other and a thickness generally orthogonal to the planar surfaces. As used herein, “planar surface” refers to surfaces that are generally recognized as flat or capable of being laid flat. For example, in any embodiment herein, a generally planar surface may include minor undulations and/or deviations. FIG. 1A, for example, illustrates an absorbent, antimicrobial layer 110 includeing a first planar surface 111 (e.g., a wound-facing surface) and a second planar surface 112 (e.g., a back surface). In any embodiment herein, the absorbent, antimicrobial layer may include planar surfaces having a surface area from about 1 cm² to about 400 cm², such as from about 2 cm² to about 200 cm² or from about 4 cm² to about 100 cm²; thus, the surface area of the absorbent, antimicrobial layer included in any embodiment herein may have a surface area of about 1 cm², about 2 cm², about 3 cm², about 4 cm², about 5 cm², about 6 cm², about 7 cm², about 8 cm², about 9 cm², about 10 cm², about 11 cm², about 12 cm², about 13 cm², about 14 cm², about 15 cm², about 16 cm², about 17 cm², about 18 cm², about 19 cm², about 20 cm², about 25 cm², about 30 cm², about 40 cm², about 50 cm², about 55 cm², about 60 cm², about 65 cm², 70 cm², about 75 cm², about 80 cm², about 85 cm², about 90 cm², about 95 cm², about 100 cm², about 150 cm², about 200 cm², about 250 cm², about 300 cm², about 350 cm², about 400 cm², or any range including and/or in between any two of the preceding values. The absorbent, antimicrobial layer may be configured to have a variety of shapes, including but not limited to square, rectangular, elliptical, pentangular, circular, or oval.

In any embodiment herein, the absorbent, antimicrobial layer may include a substrate or matrix exhibiting and/or imparting one or more desired characteristics or parameters. For example, in any embodiment herein, the absorbent, antimicrobial layer may include an antimicrobial material. The antimicrobial material may be in any suitable form as allows for incorporation of the antimicrobial material into the absorbent, antimicrobial layer, including but not limited to, antimicrobial fibers. For example, in any embodiment herein, the antimicrobial fibers may have an average length from about 0.25 inches to about 6 inches, such as from about 0.5 inches to about 4 inches or from about 0.75 inches to about 3 inches; thus, antimicrobial fibers included in any embodiment herein may have an average length of about 0.25 inches, about 0.5 inches, about 0.75 inches, about 1 inch, about 1.25 inches, about 1.50 inches, about 1.75 inches, about 2 inches, about 2.25 inches, about 2.50 inches, about 2.75 inches, about 3 inches, about 3.25 inches, about 3.50 inches, about 3.75 inches, about 4 inches, about 4.25 inches, about 4.50 inches, about 4.75 inches, about 5 inches, about 5.25 inches, about 5.50 inches, about 5.75 inches, about 6 inches, or any range including and/or in between any two of the preceding values. The antimicrobial fibers may have a denier/filament (DPF) from about 0.5 to about 40, such as from about 0.75 to about 30 DPF or from about 1 to about 10 DPF; thus, the antimicrobial fibers may have a denier included in any embodiment herein may have a denier of about 0.5 DPF, about 1 DPF, about 1.5 DPF, about 2 DPF, about 3 DPF, about 4 DPF, about 5 DPF, about 6 DPF, about 7 DPF, about 8 DPF, about 9 DPF, about 10 DPF, about 11 DPF, about 12 DPF, about 13 DPF, about 14 DPF, about 15 DPF, about 16 DPF, about 17 DPF, about 18 DPF, about 19 DPF, about 20 DPF, about 25 DPF, about 30 DPF, about 35 DPF, about 40 DPF, or any range including and/or in between any two of the preceding values.

In any embodiment herein, the antimicrobial material may include base fibers having an antimicrobial coating. The base fibers may include a synthetic material. Suitable synthetic materials may include but are not limited to polyamides, polyolefins, polyesters, polypropylenes, polyethylenes (e.g., high-molecular-weight polyethylene (HMWP)), or combinations of two or more thereof. Thus, in any embodiment herein, the base fibers may include a polyamide, such as nylon.

In any embodiment herein, the antimicrobial coating may be a metallic coating, such as a silver coating. The silver coating may include silver in metallic form, in ionic form (e.g., a silver salt, such as silver acetate and/or silver citrate), or a combination thereof. In any embodiment herein, the metallic coating may include silver in combination with one or more additional metals, including but not limited to, gold, platinum, ferro-manganese, copper, zinc, or combinations of two or more thereof.

In any embodiment herein, the absorbent, antimicrobial layer may include a safe and effective amount of the antimicrobial coating. As referred to herein, a “safe and effective” amount is an amount that is sufficient to have the desired effect (e.g., antimicrobial activity, with respect to silver), without undue adverse side effects (such as toxicity, irritation, or allergic response), commensurate with a reasonable benefit/risk ratio when used in the manner of this technology. The specific safe and effective amount of the silver or another antimicrobial coating may vary with such factors as the form of silver, the type and quantity of other materials in the matrix, the intended use, and the physical condition of the subject on whom the dressings are used.

The absorbent, antimicrobial layer may include at least about 90% of base fibers having a silver coating, such as at least about 95% of base fibers having a silver coating or about 100% of base fibers having a silver coating; thus, in any embodiment herein the amount of base fibers having a silver coating in the absorbent, antimicrobial layer may be about 90%, about 91%, about 92%, about 93%, about 94%, about 95%, about 98%, about 99%, about 100%, or any range including and/or in between any two of the preceding values. Additionally, the amount of the silver coating on the base fibers may be from about 4% to about 75% by weight of the antimicrobial fibers, such as from about 8% to about 60% by weight of the antimicrobial fibers or from about 12% to about 30% by weight of the antimicrobial fibers; thus, the amount of the silver coating on the base fibers may be (by weight of the antimicrobial fibers) about 4%, about 6%, about 8%, about 10%, about 12%, about 14%, about 16%, about 18%, about 20%, about 25%, about 30%, about 35%, about 40%, about 45%, about 50%, about 55%, about 60%, about 65%, about 70%, about 75%, or any range including and/or in between any two of the preceding values. Suitable antimicrobial fibers (e.g., base fibers having a silver coating) and methods of making the same are disclosed in U.S. Pat. No. 7,385,101, incorporated herein by reference in its entirety.

In any embodiment herein, the antimicrobial coating may include polyhexanide (also known as polyhexamethylene biguanide or PHMB), chlorhexidine, povidone iodine, triclosan, sucralfate, a quaternary ammonium salt, or a combination of any two or more thereof.

In any embodiment herein, the absorbent, antimicrobial layer may include an absorbent material. The absorbent material may include materials capable of absorbing an aqueous medium, including but not limited to water, blood, and/or wound exudate. The absorbent material may form a gel when contacted with sufficient quantities of the aqueous medium. For example, in any embodiment herein, the absorbent material may absorb the aqueous medium in an amount of at least about 10 grams per gram of the absorbent material, such as about 15 grams, 20 grams, or 25 grams per gram of the absorbent material; thus, the amount of aqueous medium absorbed per gram of the absorbent material may include in any embodiment herein about 10 grams, about 11 grams, about 12 grams, about 13 grams, about 14 grams, about 15 grams, about 16 grams, about 17 grams, about 18 grams, about 19 grams, about 20 grams, about 21 grams about 22 grams, about 23 grams, about 24 grams, about 25 grams, or any range including and/or in between any two of the preceding values. The absorbent material of any embodiment herein may be in any suitable form for incorporation within the absorbent, antimicrobial layer. For example, in any embodiment herein, the absorbent material may include a gel, a hydrogel, a fiber, a bead, a particulate, or a combination of two or more thereof. Thus, in any embodiment herein, the absorbent material may include absorbent fibers.

In any embodiment herein, the absorbent material may include a material exhibiting suitable absorption properties and that does not exhibit toxicity or immunogenicity with respect to mammals—for example, a polysaccharide. Examples of polysaccharides suitable for use as the absorbent material include hydrocolloids, cellulosic materials, hyaluronic acid, hyaluronic acid salts, or a combination of two or more thereof. Suitable hydrocolloids include, but are not limited to, alginic acids and alginic acid salts, guar gum, locust bean gum, pectin, gelatin, xanthum gum, karaya gum, chitosan, or a combination of two or more thereof. Thus, in any embodiment herein, the absorbent material may include an alginic acid salt. Suitable alginic acid salts include, but are not limited to, calcium alginate, sodium alginate, potassium alginate, or a combination of any two or more thereof. Suitable cellulosic materials include, but are not limited to, carboxymethylcellulose (CMC), CMC derivatives (e.g., sodium CMC, methylcellulose, hydroxymethylcellulose, or combinations thereof), cellulose ethyl sulphonate (CES), or a combination of any two or more thereof. In any embodiment herein, the absorbent material may include an alginate in combination with CMC, for example, calcium alginate fibers in combination with CMC fibers. Examples of suitable absorbent materials, including absorbent fibers, and methods of making the same are disclosed in U.S. Pat. No. 7,385,101, incorporated herein by reference in its entirety.

In any embodiment herein, the absorbent, antimicrobial layer may include both an antimicrobial material and an absorbent material as described herein in any embodiment. For example, the absorbent, antimicrobial layer may include a blend of an antimicrobial material and an absorbent material. The blend may a homogenous blend of the antimicrobial material and the absorbent material such that the absorbent, antimicrobial layer generally exhibits uniform or substantially uniform absorption of an aqueous medium and exhibits uniform or substantially uniform antimicrobial activity (e.g., uniform or substantially uniform release of silver ions). For example, in any embodiment herein, the absorbent, antimicrobial layer may include a blend of nylon fibers coated with a silver coating, calcium alginate fibers, and CMC fibers.

The amount of antimicrobial fibers (e.g., silver-coated nylon fibers) present within the absorbent, antimicrobial layer any embodiment herein may be about 30% to about 70% by weight of the absorbent, antimicrobial layer, such as from about 40% to about 60% by weight of the absorbent, antimicrobial layer. For example, in any embodiment herein, the amount of antimicrobial fibers by weight of the absorbent, antimicrobial layer may be about 30%, about 35%, about 40%, about 45%, about 50%, about 55%, about 60%, about 65%, about 70%, or any range including and/or in between any two of the preceding values.

The amount of absorbent fibers (e.g., calcium alginate fibers and CMC fibers) present within the absorbent, antimicrobial layer may be in an amount of about 30% to about 70% by weight of the absorbent, antimicrobial layer, such as from about 40% to about 60% by weight of the absorbent, antimicrobial layer. Thus, in any embodiment herein, the amount of absorbent fibers included in the absorbent, antimicrobial layer may be (by weight of the absorbent, antimicrobial layer) about 30%, about 35%, about 40% , about 45%, about 50%, about 55%, about 60%, about 65%, about 70%, or any range including and/or in between any two of the preceding values. In any embodiment herein, the absorbent fibers may include calcium alginate fibers and CMC fibers. The calcium alginate fibers and CMC fibers may be present at a weight ratio (calcium alginate fibers:CMC fibers) of about 1:12 to about 1:1, such as from about 1:8 to about 1:2. For example, in any embodiment herein, the calcium alginate fibers and CMC fibers may be present at a weight ratio (calcium alginate fibers:CMC fibers) of about 1:1, about 1:2, about 1:3, about 1:4, about 1:5, about 1:6, about 1:7, about 1:8, about 1:9, about 1:10, about 1:11, about 1:12, or any range including and/or in between any two of the preceding values.

In any embodiment herein, the absorbent, antimicrobial layer may include a blend, in any suitable form, of the antimicrobial material and absorbent material, for example, a nonwoven matrix or a woven matrix. The nonwoven matrix or woven matrix may have a thickness from about 1 mm to about 25 mm, such as from about 1.5 mm to about 15 mm, from about 2 mm to about 12 mm, or from about 2.5 mm to about 10 mm. Thus, in any embodiment herein, the nonwoven matrix or woven matrix of the absorbent, antimicrobial layer may have a thickness of about 1 mm, about 2 mm, about 3 mm, about 4 mm, about 5 mm, about 6 mm, about 7 mm, about 8mm, about 9 mm, about 10 mm, about 11 mm, about 12 mm, about 13 mm, about 14 mm about 15 mm, about 16 mm, about 17 mm, about 18 mm, about 19 mm, about 20 mm, about 21 mm, about 22 mm, about 23 mm, about 24 mm, about 25 mm, or any range including and/or in between any two of the preceding values.

The absorbent, antimicrobial layer may further include one or more preservatives, stabilizing agents, plasticizers, matrix strengthening materials, antioxidant dyestuffs, active materials, or a combination of two or more thereof.

Matrix strengthening materials improve handling characteristics—for example, for example, decreasing the susceptibility to tearing of the absorbent, antimicrobial layer (e.g., decreasing the susceptibility to tearing of a CMC-containing absorbent, antimicrobial layer). Non-gelling cellulose fibers are an example of a matrix strengthening material, where such “non-gelling” cellulose fibers may be substantially water insoluble and may be produced from cellulose that has not been chemically modified to increase water solubility (as contrasted from CMC or other cellulose ethers). Non-gelling cellulose fibers are commercially available (e.g., Tencel® fibers sold by Lenzing AG). The non-gelling cellulose fibers may be processed from a commercially-available continuous length, such as by cutting into lengths that are from about 0.5 cm to about 5 cm in length. Thus, in any embodiment herein, the absorbent, antimicrobial layer may include non-gelling fibers having a length of about 0.5 cm, about 0.6 cm, about 0.7 cm, about 0.8 cm, about 0.9 cm, about 1 cm, about 1.2 cm, about 1.4 cm, about 1.6 cm, about 1.8 cm, about 2 cm, about 2.2 cm, about 2.4 cm, about 2.6 cm, about 2.8 cm, about 3 cm, about 3.2 cm, about 3.4 cm, about 3.6 cm, about 3.8 cm, about 4 cm, about 4.2 cm, about 4.4 cm, about 4.6 cm, about 4.8 cm, about 5 cm, or any range including and/or in between any two of the preceding values. The non-gelling cellulose fibers may be present in the absorbent, antimicrobial layer in an amount appropriate to result in the desired physical characteristics of the absorbent, antimicrobial layer. For example, in any embodiment herein, the non-gelling cellulose fibers may be present at a level from about 5% to about 50% by weight of the absorbent, antimicrobial layer, such as from 10% to about 40% or from about 15% to about 25% by weight of the absorbent, antimicrobial layer.

As discussed above, the absorbent, antimicrobial layer may further include an antioxidant dyestuff, typically having absorbance in the visible light region (i.e., 400-700 nm) where such dyestuffs may be operable to photochemically trap generated free radicals that could otherwise react with the silver in the absorbent, antimicrobial layer, acting as photochemical desensitisers. In any embodiment herein, the antioxidant dyestuff may include aniline dyes, acridine dyes, thionine dyes, bis-naphthalene dyes, thiazine dyes, azo dyes, anthraquinone dyes, or a mixture of any two or more thereof. For example, in any embodiment herein, the antioxidant dyestuff may be gentian violet, aniline blue, methylene blue, crystal-violet, acriflavine, 9-aminoacridine, acridine yellow, acridine orange, proflavin, quinacrine, brilliant green, trypan blue, trypan red, malachite green, azacrine, methyl violet, methyl orange, methyl yellow, ethyl violet, acid orange, acid yellow, acid blue, acid red, thioflavin, alphazurine, indigo blue, methylene green, or a mixture of any two or more thereof. The antioxidant dyestuff may be included in an amount of about 0.05% to about 5% by weight of the absorbent, antimicrobial layer, such as about 0.2% to about 2% by weight of the absorbent, antimicrobial layer.

Also as discussed above, the absorbent, antimicrobial layer may further include one or more active materials. Such active materials may, for example, be effective to aid in wound healing. Exemplary active materials include, but are not limited to, a non-steroidal anti-inflammatory drug (e.g., acetaminophen), a steroid, an antibiotic (e.g., penicillins and/or streptomycins), antiseptics other than silver (e.g., chlorhexidine), growth factors (e.g., fibroblast growth factor (FGF), an epidermal growth factor (EGF), a platelet derived growth factor(PGDF)), or a combination of any two or more thereof. The active materials may be present in an amount from about 0.1% to about 10% by weight of the absorbent, antimicrobial layer, such as from about 1% to about 5% by weight of the absorbent, antimicrobial layer; thus, the amount of active material present by weight of the absorbent, antimicrobial layer may be about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, or any range including and/or in between any two of the preceding values.

The absorbent, antimicrobial layer of any embodiment herein may be a commercially-available product exhibiting the antimicrobial activity, absorbency, or a combination thereof as described herein in any embodiment. An exemplary commercially-available product is SILVERCEL™ Dressing available from Acelity in San Antonio, Tx. Additional exemplary materials which may suitably be employed as the absorbent, antimicrobial layer and methods of making the same are disclosed in U.S. Pat. No. 7,385,101, incorporated herein in its entirety.

### Bioresorbable Layer

The bioresorbable layer of the dressing may be configured to degrade at least a portion of a biofilm. As used herein, a “biofilm” refers to an association of microorganisms that are encased or embedded within a matrix material, which may be self-produced by resident microorganisms (e.g., extracellular DNA; proteins), and may further include biological films that develop and persist at interfaces in aqueous environments . The biofilm may adhere to living and/or non-living surfaces, such as tissue, wounds, medical implants (e.g., orthopedic implants, dental implants, catheters, stents, and the like). In any embodiment herein, the biofilm may include bacteria, non-bacterial microorganisms, a matrix material, or a combination of any two or more thereof. Suitable bacteria may include in any embodiment herein Gram-negative bacteria (e.g., Pseudomonas aeruginosa), Gram-positive bacteria (e.g., Staphylococcus aureus and Streptococcus mutans), or combinations thereof. Suitable non-bacterial microorganisms may include yeasts, for example, Candida albicans. Suitable matrix materials may include an extracellular polymeric substance (EPS) matrix, such as polymers (e.g., biopolymers) having relatively high molecular weights. Suitable EPS matrix may include, but are not limited to, polysaccharides, glycoproteins (e.g., lipids), nucleic acids (e.g., DNA or RNA), or combinations of two or more thereof. The EPS matrix may contribute to various properties of the biofilm. For example, the EPS matrix may impart decreased antimicrobial susceptibility to the microorganisms of the biofilm by providing a physical barrier to those antimicrobials.

In any embodiment herein, the bioresorbable layer may include an anti-biofilm agent, which is capable of degrading a biofilm. For example, in any embodiment herein, the bioresorbable layer may include an anti-biofilm agent suitable for degrading an EPS matrix, referred to herein as an EPS-active agent. Suitable EPS-active agents may include, but are not limited to, citric acid; ascorbic acid; carboxylic acids (e.g., acetic acid, formic acid, gluconic acid, lactic acid, oxalic acid, tartaric acid, and peroxy-pyruvic acid); or a combination or any two or more thereof. Thus, in any embodiment herein, the bioresorbable layer may include citric acid. The EPS-active agent may be present within the bioresorbable layer in an amount from about 0.05% to about 5.0% by weight of the bioresorbable layer, such as from about 0.5% to about 2.5% by weight of the bioresorbable layer, or from about 1.0% to about 2.0% by weight of the bioresorbable layer; thus, the amount of EPS-agent by weight to the bioresorbable layer of any embodiment may be about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, or any range including and/or in between any two of the preceding values.

The bioresorbable layer may include a bioresorbable polymer as a bioresorbable material. As used herein, “bioresorbable” or “bioresorbability” refers to a characteristic of a material to at least partially or fully disintegrate, degrade, or dissolve upon exposure to physiological fluids or processes. For example, in any embodiment herein, at least a portion of the bioresorbable layer may be absorbed or assimilated at a tissue site or in vivo (e.g., in a mammalian body). The bioresorbability may be exhibited as a result of a chemical process or condition, a physical process or condition, or combinations thereof. The bioresorbable layer may be configured to exhibit a particular proportion of disintegration, degradation, and/or dissolution (hereafter referred to as “broken down”) within a particular time period. In any embodiment herein, the bioresorbable layer may be configured such that about 90% by weight, about 91% by weight, about 92% by weight, about 93% by weight, about 94% by weight, about 95% by weight, about 96% by weight, about 97% by weight, about 98% by weight, about 99% by weight, or about 100% by weight of the bioresorbable layer (or any range including and/or in between any two of the preceding values) may be broken down within a time period of about 24 hours, about 26 hours, about 28 hours, about 30 hours, about 32 hours, about 34 hours, about 36 hours, about 38 hours, about 40 hours, about 42 hours, about 44 hours, about 46 hours, about 2 days, about 3 days, about 4 days, about 5 days, about 6 days, or about 7 days (or any range including and/or in between any two of the preceding values) from introduction into a physiological environment or when incubated with simulated physiological fluid at a temperature of about 37° C.

In any embodiment herein, the bioresorbable layer may include the bioresorbable polymer and the EPS-active agent such that the bioresorbable layer releases the EPS-active agent as the bioresorbable polymer is broken down. Suitable bioresorbable polymers may include, but are not limited to, gelatin, collagen, oxidized regenerated cellulose (ORC), polylactic acid, polyglycolic acid, a starch, or a combination of any two or more thereof. In any embodiment herein, the bioresorbable layer may include a composition that includes the EPS-active agent and the bioresorbable polymer such that, upon resorption of the bioresorbable polymer, the EPS-active agent may be released.

The collagen of the bioresorbable layer may be a mammalian collagen. Additionally or alternatively, in some embodiments, the collagen of the bioresorbable layer may comprise human collagen type I and human collagen type III. Additionally or alternatively, in some embodiments, the collagen of the bioresorbable layer may comprise bovine collagen type I and bovine collagen type III. In any embodiment disclosed herein, mammalian recombinant collagen of the bioresorbable layer may be provided by any suitable method known in the art. Additionally or alternatively, in some embodiments, human recombinant collagen of the bioresorbable layer may be provided by any suitable method known in the art. For example, the step of providing human recombinant collagen may comprise following the protocol described in U.S. Pat. No. 5,962,648, the entire content of which is incorporated herein by reference. Further recombinant processes are set forth in U.S. Pat. No. 5,593,859 and WO2004/078120 which are also incorporated herein by reference. Additionally or alternatively, in some embodiments, collagen will be recombinantly manufactured by culturing a cell which has been transfected with at least one gene encoding a polypeptide comprising collagen and genes encoding oxidized cellulose and subunits of the post-translational enzyme prolyl 4-hydroxylase, and purifying the resultant collagen monomer therefrom. The human recombinant collagen solution may be subsequently subjected to polymerization or cross-linking conditions to produce an insoluble fibrous collagen.

In any embodiment disclosed herein, the bioresorbable layer may include about 30 wt. % to about 70 wt. % collagen by weight of the bioresorbable layer. The collagen may have a weight-average molecular weight of about 5,000 to about 100,000. Thus, the collagen in the bioresorbable layer may be (by weight of the bioresorbable layer) about 30%, about 32%, about 34%, about 36%, about 38%, about 40%, about 42%, about 44%, about 46%, about 48%, about 50%, about 52%, about 54%, about 56%, about 58%, about 60%, about 62%, about 64%, about 66%, about 68%, about 70%, or any range including and/or in between any two of these values. The collagen in the bioresorbable layer may have a weight-average molecular weight of about 5,000, about 6,000, about 7,000, about 8,000, about 9,000, about 10,000, about 12,000, about 14,000, about 16,000, about 18,000, about 20,000, about 22,000, about 24,000, about 26,000, about 28,000, about 30,000, about 32,000, about 34,000,about 36,000, about 38,000, about 40,000, about 45,000, about 50,000, about 55,000, about 60,000, about 65,000, about 70,000, about 75,000, about 80,000, about 85,000, about 90,000, about 95,000, about 100,000, or any range including and/or in between any two of these values.

In any embodiment disclosed herein, the collagen of the bioresorbable layer may include a weight ratio of human collagen type Ito human collagen type III of about 100:0, about 90:10, about 80:20, about 70:30, about 60:40, about 50:50, about 40:60, about 30:70, about 20:80, about 10:90, about 0:100, or any range including and/or in between any two of these values. Additionally or alternatively, in some embodiments, the ratio by weight of human collagen type Ito human collagen type III is greater than about 50:50, or greater than about 70:30. Additionally or alternatively, in some embodiments, the collagen of the bioresorbable layer may include a weight ratio of type I bovine collagen to type III bovine collagen of about 85:15.

In any embodiment disclosed herein, ORC may be produced by the oxidation of cellulose, for example with dinitrogen tetroxide and/or as described in U.S. Pat. No. 3,122,479 (incorporated herein by reference). Without wishing to be bound by theory, it is believed that this process may convert primary alcohol groups on the saccharide residues of the cellulose to carboxylic acid groups, for example, forming uronic acid residues within the cellulose chain. The oxidation may not proceed with complete selectivity, and as a result hydroxyl groups on carbons 2 and 3 of the saccharide residue may be converted to the keto form. These ketone units may introduce an alkali labile link, which at pH 7 or higher initiates the decomposition of the polymer via formation of a lactone and sugar ring cleavage. As a result, oxidized regenerated cellulose is biodegradable and bioresorbable under physiological conditions. ORC is available with a variety of degrees of oxidation and hence rates of degradation. The ORC may include particles, fibers, or both; in any embodiment disclosed herein, the ORC may be in the form of particles, such as fiber particles or powder particles. In embodiments that include ORC fibers, the ORC fibers may have a volume fraction such that at least 80% of the fibers have lengths in the range from about 5 μm to about 1000 μm, or from about 250 μm to about 450 μm.

In any embodiment disclosed herein, the bioresorbable layer may include from about 30% to about 70% of the ORC by weight of the bioresorbable layer; thus the bioresorbable layer may include ORC in an amount (by weight of the bioresorbable layer) of about 30%, about 32%, about 34%, about 36%, about 38%, about 40%, about 42%, about 44%, about 46%, about 48%, about 50%, about 52%, about 54%, about 56%, about 58%, about 60%, about 62%, about 64%, about 66%, about 68%, about 70%, or any range including and/or in between any two of these values.

In any embodiment disclosed herein, the bioresorbable layer may include from about 30 wt. % to about 90 wt. % of the mixture of collagen and ORC. Thus, the amount of the collagen and ORC mixture in the bioresorbable layer may be about 30%, about 32%, about 34%, about 36%, about 38%, about 40%, about 42%, about 44%, about 46%, about 48%, about 50%, about 52%, about 54%, about 56%, about 58%, about 60%, about 62%, about 64%, about 66%, about 68%, about 70%, about 72%, about 74&, about 76%, about 78%, about 80%, about 82%, about 84%, about 86%, about 88%, about 90%, or any range including and/or in between any two of these values.

In any embodiment disclosed herein, the bioresorbable layer may include collagen (of any embodiment disclosed herein) and ORC (of any embodiment disclosed herein), where a weight ratio of collagen to ORC in the bioresorbable layer is about 60:40 to about 40:60; thus, the weight ratio of collagen to ORC in the bioresorbable layer may be about 60:40, about 59:41, about 58:42, about 57:43, about 56:44, about 55:45, about 54:46, about 53:47, about 52:48, about 51:49, about 50:50, about 49:51, about 48:52, about 47:53, about 46:54, about 45:55, about 44:56, about 43:57, about 42:58, about 41:59, about 40:60, or any range including and/or in between any two of these values.

The bioresorbable layer may further include a plasticizer, such as glycerol or other polyhydric alcohol. For example, in any embodiment herein, the plasticizer may be present in the bioresorbable layer in amount from about 0.1% to about 2.0%, such as from about 0.5% to about 1.5% by weight of the bioresorbable layer. Suitable amounts of plasticizer by weight of the bioresorbable layer may include, but are not limited to, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, or any range including and/or in between any two of the preceding values.

The bioresorbable layer may be a film having a suitable thickness from about 100 μm to about 3000 μm; thus, in any embodiment herein, the film may have a thickness of about 100 μm, about 200 μm, about 300 μm, about 400 μm, about 500 μm, about 600 μm, about 700 μm, about 800 μm, about 900 μm, about 1000 μm, about 1100 μm, about 1200 μm, about 1300 μm, about 1400 μm, about 1500 μm, about 1600 μm, about 1700 μm, about 1800 μm, about 1900 μm, about 2000 μm, or any range including and/or in between any two of the preceding values. In any embodiment herein, the bioresorbable layer may be a film characterized as impermeable to fluids or substantially impermeable to fluids (e.g., a non-porous film). In any embodiment herein, the bioresorbable layer may be a film configured to allow the passage of a fluid. For example, the bioresorbable layer may include a plurality of pores extending there-through so as to allow fluid communication through the bioresorbable layer. The plurality of pores may have an average pore size in a range from about 200 μm to about 3000 μm; thus, the average pore size in any embodiment herein may be about 200 μm, about 300 μm, about 400 μm, about 500 μm, about 600 μm, about 700 μm, about 800 μm, about 900 μm, about 1000 μm, about 1200 μm, about 1400 μm, about 1600 μm, about 1800 μm, about 2000 μm, about 2200 μm, about 2400 μm, about 2600 μm, about 2800 μm, about 3000 μm, or any range including and/or in between any two of the preceding values. The bioresorbable layer may include a plurality of pores at a pore density of about 2 pores/cm² to about 10 pores/cm²; thus, the bioresorbable layer of any embodiment herein may have a pore density of about 2 pores/cm², about 4 pores/cm², about 6 pores/cm², about 8 pores/cm², about 10 pores/cm², or any range including and/or in between any two of the preceding values.

In any embodiment herein, the bioresorbable layer may be applied to one or more suitable surfaces of the absorbent, antimicrobial layer, such that the bioresorbable layer covers the one or more surfaces of the absorbent, antimicrobial layer. In one exemplary embodiment, FIG. 1A illustrates that the bioresorbable layer 120 may be applied, for example, laminated as a film, to the first planar surface 111 (e.g., the wound-facing surface).

### Non-Adherent Layer

In any embodiment herein, the dressing may include a non-adherent layer configured to be substantially non-adherent with respect to tissue at a tissue site. For example, in any embodiment herein, the non-adherent layer may be configured for contact with a tissue site. In any embodiment herein, the non-adherent layer may include a generally flexible, substantially non-adherent material. Exemplary non-adherent materials include, but are not limited to, thermoplastic materials thermoplastic elastomers, or a combination thereof. Suitable thermoplastic materials include, but are not limited to acrylics and/or acrylates (e.g., ethyl methacrylate (EMA)). Suitable thermoplastic elastomers may include, but are not limited to, nylon, styrene ethylene butene styrene (SEBS), and other block copolymers (e.g., polyether block polyamide (PEBAX)), silicone elastomers, poly caprolactam, poly lactic acid, and polyolefins (e.g., polyethylene and polypropylene)).

The non-adherent layer may be a woven material or a nonwoven material (e.g., a film). The non-adherent layer may have a thickness of about 10 μm, about 20 μm, about 30 μm, about 40 μm, about 50 μm, about 60 μm, about 70 μm, about 80 μm, about 90 μm, 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, or any range including and/or in between any two of the preceding values.

The non-adherent layer may be configured such that at least a portion of the non-adherent layer may be permeable to various wound fluids (e.g., water, blood, or wound exudate). For example, in any embodiment herein, the non-adherent layer may be porous, perforated, or a combination thereof, such that the non-adherent layer includes a plurality of pores extending there-through to allow fluid communication through the non-adherent layer. The plurality of pores may have an average pore size of about 200 μm, about 300 μm, about 400 μm, about 500 μm, 600 μm, about 700 μm, about 800 μm, about 900 μm, about 1000 μm, about 1200 μm, about 1400 μm, about 1600 μm, about 1800 μm, about 2000 μm, about 2200 μm, about 2400 μm, about 2600 μm, about 2800 μm, about 3000 μm, or any range including and/or in between any two of the preceding values. In any embodiment herein, the non-adherent layer may include a plurality of pores at a pore density of about 2 pores/cm² to about 10 pores/cm²; thus, the non-adherent layer may have a pore density of about 2 pores/cm², about 4 pores/cm², about 6 pores/cm², about 8 pores/cm², about 10 pores/cm², or any range including and/or in between any two of the preceding values.

In any embodiment herein, the non-adherent layer may be configured to cover one or more surfaces of the bioresorbable layer, the absorbent, antimicrobial layer, or a combination thereof. For example, as illustrated in FIG. 1A, the non-adherent layer 130 may cover or be applied to an outer surface of the bioresorbable layer 120. In any embodiment herein, the non-adherent layer may be laminated as a film or sheet to a generally planar surface of the bioresorbable layer opposite the surface of the bioresorbable layer that faces the absorbent, antimicrobial layer.

### Dressing Configurations

In any embodiment herein, the bioresorbable layer may be applied to a second planar surface of the absorbent, antimicrobial layer. For example, as illustrated in FIG. 1B, a first bioresorbable layer 120a may be applied to a first planar surface 111, and a second bioresorbable layer 120b may be applied (e.g., laminated as a film) to a second planar surface 112 of the absorbent, antimicrobial layer 110.

The non-adherent layer may be applied to various surfaces of the bioresorbable layer, the absorbent, antimicrobial layer, or a combination thereof. For example, by reference to an illustrative exemplary embodiment in FIG. 1B, a first non-adherent layer 130a may be applied (e.g., laminated as a film) to the first bioresorbable layer 120a, and a second non-adherent layer 130b may be applied (e.g., laminated as a film) to the second bioresorbable layer 120b. In another exemplary embodiment illustrated in FIG. 1C, a first non-adherent layer 130a may be applied (e.g., laminated as a film) to the bioresorbable layer 120 opposite a first planar surface 111 of the absorbent, antimicrobial layer 110, and a second non-adherent layer 130b may be applied (e.g., laminated as a film) to the second planar surface 112 of the absorbent, antimicrobial layer 110.

The non-adherent layer may include an envelope; for example, in any embodiment herein, the envelope may include in any suitable shape a sack, a packet, a pouch, a tube, a cylinder, a box, or a combination of any two or more thereof. For example, as illustrated in the exemplary embodiment of FIG. 1D, a first and a second non-adherent layer, 130a and 130b, together may form an envelope 135. The envelope may be formed from one or more non-adherent layers in the form of films or sheets, which may have any suitable size or shape. For example, in any embodiment herein, one or more non-adherent layers may be in the form of films or sheets that each independently may be square, rectangular, circular, oval, or oblong. In any embodiment herein, a non-adherent layer in the form of an envelope may enclose or encompass the absorbent, antimicrobial layer where the bioresorbable layer may be applied to one or more layers thereof.

In any embodiment herein, one or more of the absorbent, antimicrobial layer, the bioresorbable layer, the non-adherent layer, or combinations thereof of the dressing may be configured to be in contact with a tissue site. For example, in any embodiment herein, the absorbent, antimicrobial layer, the bioresorbable layer, the non-adherent layer, or a combination of any two or more thereof may be configured to be in contact with a portion of a tissue site, substantially all of the tissue site, or the tissue site in its entirety. In any embodiment herein, the tissue site may be a wound and the dressing may partially or completely fill the wound, or may be placed over (e.g., superior to) the wound. In any embodiment herein, the dressing may be configured to have any form, size, shape, and/or thickness suitable for a variety of treatment factors, such as the type of treatment being implemented or the nature and size of a tissue site. For example, in any embodiment herein, the size and shape of the dressing may be adapted to the contours of deep and irregular shaped tissue sites and/or may be configured so as to be adaptable to a given shape or contour. In any embodiment herein, any or all of the surfaces of one or more of the adsorbent, antimicrobial layer, bioresorbable layer, non-adherent layer, or a combination of any two or more thereof may include projections and/or an uneven, course, or jagged profile that may induce strains and stresses on a tissue site, which may be effective to promote granulation at the tissue site.

### Optional Additional Components or Layers

In any embodiment herein, the dressing may include one or more additional layers. The one or more additional layers may be configured to perform any of a variety of functions including, for example, adherence of the dressing to the wound or to surrounding tissues, increasing structural rigidity of the dressing, protection of the dressing from moisture or other materials in the external environment, protection of a wound surface, delivery of one or more active materials or other materials to the wound surface, or a combination of two or more thereof. In any embodiment herein, the additional layers may be conformable to a wound surface and/or to the surrounding tissues. For example, in any embodiment herein, the one or more additional layers may be configured to be capable of bending such that the wound-facing surfaces of the dressing are in substantial contact with the wound and/or the surrounding tissues.

In any embodiment herein, the dressing may further include a cover. For example, as illustrated in an exemplary embodiment in FIG. 2, the dressing 100 may further include a cover 220 having a first (e.g., wound-facing) surface 221 and a second (e.g., back) surface 222. The cover may support various components of the dressing, such as a surface (e.g., a back surface) of the absorbent, antimicrobial layer, the bioresorbable layer, or the non-adherent layer may be in contact with and adhered to a surface (e.g., a wound-facing surface) of the cover.

In any embodiment herein, the cover may be permeable to water vapor and impermeable or substantially impermeable to liquid. For example, in any embodiment herein, the cover is not permeable to liquid water or wound exudate. The cover may have a moisture vapor transmission rate (MVTR) of about 300 g/m²/24 hours to about 5000 g/m²/24 hours, such as from about 500 g/m²/24 hours to about 2000 g/m²/24 hours at 37.5° C. at 50% relative humidity difference as described in ASTM E96-00; thus, the MVTR may be about 500 g/m²/24 hours, about 750 g/m²/24 hours, about 1000 g/m²/24 hours, about 1500 g/m²/24 hours, about 2000 g/m²/24 hours, about 2500 g/m²/24 hours, about 3000 g/m²/24 hours, about 3500 g/m²/24 hours, about 4000 g/m²/24 hours, about 4500 g/m²/24 hours, about 5000 g/m²/24 hours, or any range including and/or in between any two of the preceding values. In any embodiment herein, the cover may be impermeable or substantially impermeable to microorganisms.

The cover may be formed from polymers. Suitable polymers for forming the cover may include, but are not limited to, polyurethanes, poly alkoxyalkyl acrylates, methacrylates, or a combination of any two or more thereof. For example, in any embodiment herein, the cover may include a continuous layer of a high-density blocked polyurethane foam that is predominantly closed-cell. Suitable cover materials (e.g., polymers) are disclosed in U.S. Pat. No. 3,645,835 issued Feb. 29, 1972, incorporated by reference herein in its entirety. In any embodiment herein, the cover material may be the polyurethane film commercially available as Estane® 5714F (sold by The Lubrizol Corporation).

The cover may have a thickness in the range from about 10 μm to about 1000 μm, such as from about 100 μm to about 500 μm; thus, the cover may have a thickness of about 10 μm, about 20 μm, about 30 μm, about 40 μm, about 50 μm, about 60 μm, about 70 μm, about 80 μm, about 90 μm, 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, or any range including and/or in between any two of the preceding values. The surfaces of the cover may have a size and configuration such that the cover extends beyond the dressing and defines a marginal region extending from about 0.5 mm to about 60 mm, such as from about 1 mm to about 50 mm; thus, in any embodiment herein, the marginal region may extend from about 0.5 mm, about 1 mm, about 2 mm, about 3 mm, about 4 mm, about 5 mm, about 6 mm, about 7 mm, about 8 mm, about 9 mm, about 10 mm, about 15 mm, about 20 mm, about 25 mm, about 30 mm, about 35 mm, about 40 mm, about 45 mm, about 50 mm, about 55 mm, about 60 mm, or any range including and/or in between any two or more of the preceding values. In any embodiment herein, the cover may extend beyond one or more edges of the dressing, including the absorbent, antimicrobial layer, the bioresorbable layer, the non-adherent layer, or a combination of any two or more thereof, on the cover. In any embodiment herein, the cover may be configured such that a portion of the marginal area around the dressing of the cover may be coated with an adhesive layer, such that when applied to a wound tissue, the marginal area may be used to adhere the dressing to tissues surrounding the wound or other tissue site.

Suitable adhesives may include, but are not limited to, pressure sensitive adhesives. For example, in any embodiment herein, the pressure sensitive adhesive may include acrylate ester copolymers, polyvinyl ethyl ether, polyurethane, or a combination of any two or more thereof. Suitable pressure sensitive adhesives include, but are not limited to, those pressure sensitive adhesives disclosed in U.S. Pat. No. 3,645,835, issued Feb. 29, 1972, incorporated herein by reference in its entirety. The adhesive layer may have a basis weight of about 20 g/m² to about 250 g/m².

In any embodiment herein, the dressing may further include a secondary layer positioned between the dressing and the cover. The secondary layer may include fluid pathways interconnected so as to improve distribution or collection of fluids. For example, in any embodiment herein, the secondary layer may be a porous foam material having a plurality of interconnected cells, pores, edges, walls, or a combination of two or more thereof to form interconnected fluid pathways (e.g., channels). Suitable porous foam materials may include, but are not limited to, cellular foam, open-cell foam, reticulated foam, porous tissue collections, other porous materials (e.g., gauze or felted mat), or a combination of two or more thereof. In any embodiment herein, the secondary layer may be a foam having pore sizes in of about 400 microns, about 420 microns, about 440 microns, about 460 microns, about 480 microns, about 500 microns, about 520 microns, about 540 microns, about 560 microns, about 580 microns, about 600 microns, or any range including and/or in between any two of the preceding values. Thus, in any embodiment herein, the secondary layer may be an open-cell, reticulated polyurethane foam.

The secondary layer may be characterized as exhibiting absorbency. For example, in any embodiment herein, the secondary layer may exhibit an absorbency of at least about 3 g saline/g, at least about 4 g saline/g, at least about 5 g saline/g, at least about 6 g saline/g, at least about 7 g saline/g, at least about 8 g saline/g, at least about 9 g saline/g, at least about 10 g saline/g, at least about 11 g saline/g, at least about 12 g saline/g, at least about 13 g saline/g, at least about 14 g saline/g, at least about 15 g saline/g, at least about 16 g saline/g, at least about 17 g saline/g, at least about 18 g saline/g, at least about 19 g saline/g, at least about 20 g saline/g, or any range including and/or in between any two of the preceding values. The secondary layer may be hydrophilic and configured to absorb (e.g., wick) fluid away from the dressing. For example, in any embodiment herein, the wicking properties of the secondary layer may draw fluid away from the dressing by capillary flow or other wicking mechanisms. Suitable hydrophilic foams include, but are not limited to, a polyvinyl alcohol, open-cell foam, hydrophilic foams made from polyether, hydrophobic foams that have been treated or coated to provide hydrophilicity, or a combination of any two or more thereof.

### Negative-Pressure Therapy

The dressing of any embodiment described herein may be employed in therapy in which a tissue site is treated with reduced pressure. Treatment of tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and/or micro-deformation of tissue at a wound site. Together, these benefits may increase development of granulation tissue and reduce healing times.

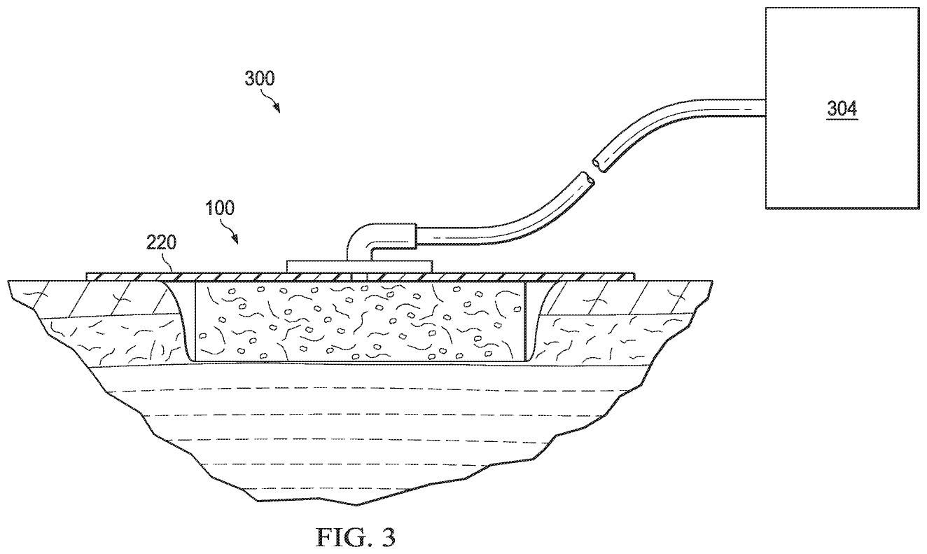

FIG. 3 illustrates an exemplary system (300) for negative-pressure therapy in a simplified schematic. Generally, the system may be configured to provide negative-pressure to a tissue site in accordance with this specification. In any embodiment herein, the system may generally include a negative-pressure supply, and may include or be configured to be coupled to a distribution component. In general, a distribution component may refer to any complementary or ancillary component configured to be fluidly coupled to a negative-pressure supply in a fluid path between a negative-pressure supply and a tissue site. In the illustration provided in FIG. 3, the dressing 100 is an example of a distribution component fluidly coupled to a negative-pressure source 304 such that negative pressure may be applied to a tissue site via dressing 100.

In any embodiment herein, the dressing may be configured to distribute negative pressure. The dressing may comprise or be configured as a manifold. A “manifold” in this context generally includes any composition or structure providing a plurality of pathways configured to collect or distribute fluid across a tissue site under pressure. For example, a manifold may be configured to receive negative pressure from the negative-pressure source and to distribute negative pressure through multiple apertures (e.g., pores), which may have the effect of collecting fluid and drawing the fluid toward the negative-pressure source. More particularly, as illustrated in FIG. 3, the dressing (100) may be configured to receive negative pressure from the negative-pressure source (304) and to distribute the negative pressure through the dressing (100), for example, which may have the effect of collecting fluid from a sealed space by drawing fluid from the tissue site through the dressing. Additionally or alternatively, the fluid path(s) may be reversed or a secondary fluid path may be provided to facilitate movement of fluid across a tissue site. Additionally or alternatively, the fluid pathways of a manifold may be interconnected to improve distribution or collection of fluids. Additionally or alternatively, a manifold may be a porous material having a plurality of interconnected cells or pores. For example, in any embodiment herein, open-cell foams such as reticulated foams may generally include pores, edges, and/or walls that may form interconnected fluid pathways (such as channels).

The fluid mechanics associated with using a negative-pressure source to reduce pressure in another component or location, such as within a sealed therapeutic environment, can be mathematically complex. However, the basic principles of fluid mechanics applicable to negative-pressure therapy are generally well-known to those skilled in the art. The process of reducing pressure may be described generally and illustratively herein as “delivering,” “distributing,” or “generating” negative pressure, for example.

In general, a fluid, such as wound fluid (for example, wound exudates and other fluids), flows toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. This orientation is generally presumed for purposes of describing various features and components herein. However, the fluid path may also be reversed in some applications (such as by substituting a positive-pressure source for a negative-pressure source) and this descriptive convention should not be construed as a limiting convention.

“Negative pressure” may generally refer to a pressure less than a local ambient pressure, such as the ambient pressure in a local environment external to a sealed therapeutic environment provided by the dressing. In many cases, the local ambient pressure may also be the atmospheric pressure proximate to or about a tissue site. Alternatively or additionally, the pressure may be less than a hydrostatic pressure associated with the tissue at the tissue site. While the amount and nature of negative pressure applied to a tissue site may vary according to therapeutic requirements, the pressure is generally a low vacuum, also commonly referred to as a rough vacuum, between −5 mm Hg (−667 Pa) and −500 mm Hg (−66.7 kPa), gauge pressure. Common therapeutic ranges are between −50 mm Hg (−6.7 kPa) and −300 mm Hg (−39.9 kPa), gauge pressure.

Additionally or alternatively, in any embodiment herein, a negative-pressure supply (such as negative-pressure source 304 of FIG. 3) may be a reservoir of air at a negative pressure, or may be a manual or electrically-powered device that can reduce the pressure in a sealed volume, such as a vacuum pump, a suction pump, a wall suction port available at many healthcare facilities, or a micro-pump, for example. A negative-pressure supply may be housed within or used in conjunction with other components, such as sensors, processing units, alarm indicators, memory, databases, software, display devices, or user interfaces that further facilitate therapy. A negative-pressure source (e.g., negative-pressure source 304 of FIG. 3) may be combined with a controller and other components into a therapy unit. A negative-pressure supply may also have one or more supply ports configured to facilitate coupling and de-coupling of the negative-pressure supply to one or more distribution components.

In any embodiment herein, components may be fluidly coupled to each other to provide a path for transferring fluids (i.e., liquid and/or gas) between the components. For example, components may be fluidly coupled through a fluid conductor, such as a tube. As used herein, the term “fluid conductor” may include a tube, pipe, hose, conduit, or other structure with one or more lumina or open passages adapted to convey a fluid between two ends thereof. Typically, a fluid conductor may be an elongated, cylindrical structure with some flexibility, but the geometry and rigidity may vary. Additionally or alternatively, in any embodiment herein, the negative-pressure source may be operatively coupled to the dressing via a dressing interface. For example, and by way of reference to FIG. 3, dressing 100 may be coupled to negative-pressure source 304 via a dressing interface such that dressing 100 receives negative pressure from negative-pressure source 304.

### Methods

The present technology also provides a therapy method, where the therapy method includes positioning a dressing of any embodiment herein of the present technology adjacent to the tissue site. For example, in any embodiment herein, the dressing may be positioned proximate to the wound. The dressing may be used with any of a variety of wounds, such as those occurring from trauma, surgery, or disease. For example, the dressing may be placed within, over, on, or otherwise proximate to the tissue site. In embodiments where the dressing includes a cover, the cover may be placed over the absorbent, antimicrobial layer, the bioresorbable layer, the non-adherent layer, or a combination or any two or more thereof, of the dressing, and the cover sealed to an attachment surface near the tissue site. For example, the dressing may be sealed to undamaged epidermis peripheral to a tissue site. In any embodiment herein, the absorbent, antimicrobial layer, the bioresorbable layer, the non-adherent layer, or a combination or any two or more thereof, may be positioned first and then the cover may be positioned proximate to the tissue site. In any embodiment herein, the absorbent, antimicrobial layer, the bioresorbable layer, the non-adherent layer, or combinations thereof and cover may be preassembled, for example, such that the absorbent, antimicrobial layer, the bioresorbable layer, the non-adherent layer, a combination or any two or more thereof, and cover may be positioned with respect to each other prior to placement proximate the tissue site. Thus, the dressing may provide a sealed therapeutic environment proximate to a tissue site, substantially isolated from the external environment.

In any embodiment herein, the process may include employing the dressing in the context of a negative-pressure therapy, where the negative-pressure therapy may include positioning the dressing proximate to the tissue site (e.g., a wound). For example, the various components of the dressing may be positioned with respect to the tissue site sequentially or, alternatively, may be positioned with respect to each other and then positioned with respect to the tissue site. The negative-pressure therapy may further comprise sealing the cover to tissue surrounding the tissue site to form a sealed space. For example, the cover may be placed over the absorbent, antimicrobial layer, the bioresorbable layer, the non-adherent layer, or a combination of any two or more thereof, and sealed to an attachment surface near the tissue site, for example, to undamaged epidermis peripheral to a tissue site.

The negative-pressure therapy method in any embodiment herein may further include fluidly coupling a negative-pressure source to the sealed space and operating the negative-pressure source to generate a negative pressure in the sealed space. For example, the negative-pressure source may be coupled to the dressing such that the negative-pressure source may be used to reduce the pressure in the sealed space. For example, negative pressure applied across the tissue site, for example, via the dressing may be effective to induce macrostrain and microstrain at the tissue site, as well as remove exudates and other fluids from the tissue site.

### Advantages

The present technology provides significant advantages, for example, when used in a wound therapy regime.

For example, conventional attempts to control biofilms during wound-healing may be made difficult by the production of the EPS matrix, which may make up at least a portion of the matrix forming a biofilm, anchor the biofilm to various wound surfaces, physically protect the bacterial cells, or combinations of these. Without wishing to be bound by theory, it is believed that a dressing of the present technology may be effective to control a biofilm, for example, by disrupting or degrading the EPS matrix. Thus, the bioresorbable layer may be effective to lower the pH in the proximity of the biofilm and disrupt the EPS matrix, thereby exposing the bacteria within the EPS matrix and rendering those bacteria susceptible to the antimicrobial activity of the absorbent, antimicrobial layer. For example, and not intending to be bound by theory, by disrupting the EPS matrix, the bioresorbable layer may improve the antimicrobial activity of the absorbent, antimicrobial layer relatively more effective in comparison to when used in the absence of the bioresorbable layer. In any embodiment herein, the dressings of the present technology may include an improved dressing, relative to a dressing including an absorbent, antimicrobial layer in the absence of an EPS-active agent. For example, the improvement may include a layer comprising the EPS-active agent, such as the bioresorbable layer. The dressings described herein in any embodiment may be used in a method for reducing microbial concentration of a biofilm. The method includes applying a dressing to a wound site. In any embodiment herein, the concentration of the microbes may be reduced by at least about 1 log, about 2 log, about 3 log, or any range including and/or in between any two of these values in comparison to the reduction in the concentration of the microbes resulting from the absorbent, antimicrobial layer alone.

The examples herein are provided to illustrate advantages and benefits of the present technology and to further assist a person of ordinary skill in the art with preparing or using the dressings of the present technology. The examples herein are also presented in order to more fully illustrate the preferred aspects of the present technology. The examples should in no way be construed as limiting the scope of the present technology, as defined by the appended claims. The examples can include or incorporate any of the variations, aspects or embodiments of the present technology described above. The variations, aspects or embodiments described above may also further each include or incorporate the variations of any or all other variations, aspects or embodiments of the present technology.

### EXAMPLES

2 grams of gelatin powder was weighed and added to 100 ml of deionized water in a beaker and mixed to form a solution. 1 gram of citric acid was added to the solution to make a 1% citric acid solution. This process was repeated using 2 grams of citric acid to make a 2% citric acid solution. Glycerol was added to each of the 1% citric acid solution and the 2% citric acid solution at a concentration of 25 μl as a plasticizer. Each of the solutions was mixed for an additional 5 minutes. 31 grams of each of the 1% citric acid solution and the 2% citric acid solution was added to a 10 cm by 10 cm square petri dish and dried overnight, yielding films having varying concentrations of citric acid. The resultant films were applied to an antimicrobial substrate (particularly, SILVERCEL™, available from Acelity) to yield various dressings, particularly a dressing including SILVERCEL™+1% citric acid and a dressing including SILVERCEL™+2% citric acid. The capability of these dressings to target bacteria was assessed using a zone of inhibition assay in which the dressings were exposed to petri dishes having a lawn of bacteria already present. The bacteria were allowed to grow and the antimicrobial activity of the respective dressings was assessed.

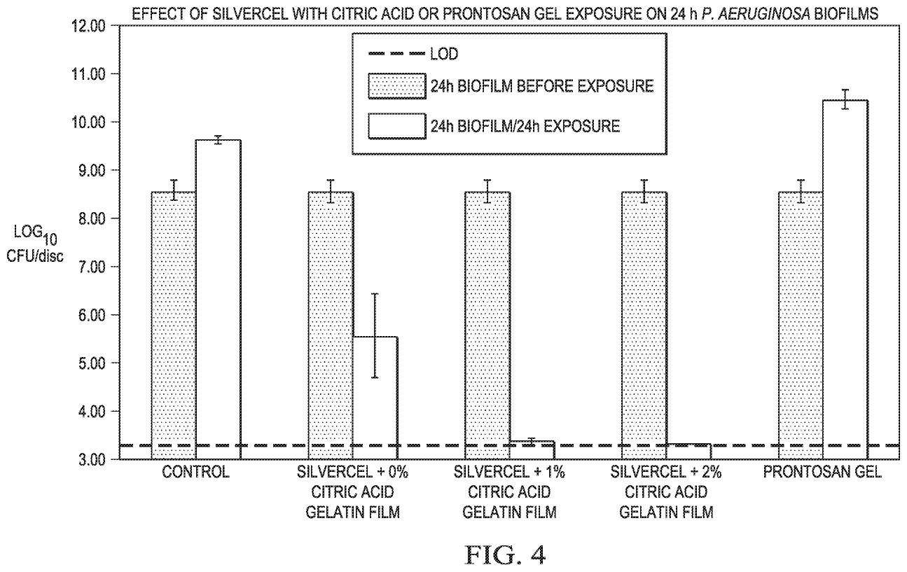

Referring to FIG. 4, the effect on biofilms of the films containing citric acid in combination with an antimicrobial substrate (particularly, the SILVERCEL™ dressing available from Acelity) is illustrated. More particularly, FIG. 4 shows that biofilm populations (particularly, P. aeruginosa biofilms) of 10⁹ CFU/disc were present prior to exposure to the various trials. The biofilm populations were assessed after 24 hours for each of the dressings, particularly, (i) a Control, (ii) SILVERCEL™ alone (0% citric acid), (iii) SILVERCEL™+1% citric acid, (iv) SILVERCEL™+2% citric acid, and (v) Prontosan Gel, a gel biofilm.

As shown SILVERCEL™ alone (0% citric acid) gave a reduction of approximately 3 log; however, the addition of citric acid yielded a significantly improved reduction in total viable count of the biofilm populations, more particularly, a reduction of more than 4 log to the detectable limits.

### EQUIVALENTS

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such claims:
A. A dressing comprising
   an absorbent, antimicrobial layer comprising nonwoven fibers; and
   a bioresorbable layer comprising an extracellular polymeric substance (EPS)-active agent.
B. The dressing of Paragraph A, wherein the absorbent, antimicrobial layer comprises an alginate.
C. The dressing of Paragraph A or Paragraph B, wherein the absorbent, antimicrobial layer comprises carboxymethylcellulose.
D. The dressing of any one of Paragraphs A-C, wherein at least a portion of the nonwoven fibers comprise nylon fibers having an antimicrobial coating.
E. The dressing of Paragraph D, wherein the antimicrobial coating comprises silver.
F. The dressing of any one of Paragraphs A-E, wherein at least a portion of the nonwoven fibers comprise absorbent fibers.
G. The dressing of Paragraph F, wherein the absorbent fibers comprise a cellulosic material.
H. The dressing of Paragraph F or Paragraph G, wherein the absorbent fibers comprise carboxymethylcellulose fibers and alginate fibers.
I. The dressing of any one of Paragraphs F-H, wherein the absorbent fibers comprise cellulose ethyl sulphonate fibers.
J. The dressing of any one of Paragraphs A-I, wherein the EPS-active agent comprises citric acid.
K. The dressing of Paragraph J, wherein the EPS-active agent comprises from 0.1% to 5% citric acid.
L. The dressing of Paragraph J or Paragraph K, wherein the EPS-active agent comprises from 0.5% to 2.5% citric acid.
M. The dressing of any one of Paragraphs J-L, wherein the citric acid is incorporated within the bioresorbable layer.
N. The dressing of any one of Paragraphs A-M, wherein the bioresorbable layer comprises gelatin, collagen, oxidized regenerated cellulose (ORC), or a combination of any two or more thereof
O. The dressing of any one of Paragraphs A-N, wherein the bioresorbable layer is a film.
P. The dressing of any one of Paragraphs A-O, further comprising a non-adherent layer, wherein the bioresorbable layer is positioned between the absorbent, antimicrobial layer and the non-adherent wound contact layer.
Q. The dressing of Paragraph P, wherein the non-adherent wound contact layer comprises ethyl methacrylate.
R. The dressing of Paragraph P or Paragraph Q, wherein the non-adherent wound contact layer is a film.
S. The dressing of any one of Paragraphs A-R, the dressing further comprising a cover positioned over the absorbent, antimicrobial layer, the bioresorbable layer, and the non-adherent wound contact layer and configured to seal to tissue surrounding the tissue site.
T. A system for providing therapy to a tissue site, the system comprising a dressing of any one of Paragraphs A-S.
U. A method for providing therapy to a tissue site, the method comprising positioning a dressing of any one of Paragraphs A-S adjacent to the tissue site.
V. The method of Paragraph U, further comprising providing negative pressure to the tissue site.
W. A dressing comprising
   an absorbent, antimicrobial layer formed from nonwoven fibers;
   a bioresorbable layer configured to degrade at least a portion of a biofilm; and
   a non-adherent wound contact layer;
   wherein the bioresorbable layer is positioned between the absorbent, antimicrobial layer and the non-adherent wound contact layer.
X. The dressing of Paragraph W, wherein the bioresorbable layer comprises citric acid.
Y. The dressing of Paragraph X, wherein the bioresorbable layer contains between 0.1% and 5% of citric acid.
Z. The dressing of Paragraph X or Paragraph Y, wherein the bioresorbable layer contains between 0.5% and 2.5% of citric acid.
AA. The dressing of any one of Paragraphs X-Z, wherein the absorbent, antimicrobial layer comprises an alginate.
AB. The dressing of any one of Paragraphs X-AA, wherein the absorbent, antimicrobial layer comprises carboxymethylcellulose.
AC. A system for providing therapy to a tissue site, the system comprising a dressing of any one of Paragraphs X-AB.
AD. A method for providing therapy to a tissue site, the method comprising positioning a dressing of any one of Paragraphs X-AB adjacent to the tissue site.
AE. The method of Paragraph AD, further comprising providing negative pressure to the tissue site.
AF. A dressing comprising
   a first layer comprising absorbent fibers and antimicrobial fibers; and
   a second layer comprising a bioresorbable material and an extracellular polymeric substance (EPS)-active agent.
AG. The dressing of Paragraph AF, wherein the absorbent fibers comprise alginate fibers.
AH. The dressing of Paragraph AF or Paragraph AG, wherein the absorbent fibers comprise carboxymethylcellulose.
AI. The dressing of any one of Paragraphs AF-AH, wherein the antimicrobial fibers comprise nylon fibers having an antimicrobial coating.
AJ. The dressing of Paragraph AI, wherein the antimicrobial coating comprises silver.
AK. The dressing of any one of Paragraphs AF-AJ, wherein the absorbent fibers comprise a cellulosic material.
AL. The dressing of any one of Paragraphs AF-AK, wherein the absorbent fibers comprise carboxymethylcellulose fibers and alginate fibers.
AM. The dressing of any one of Paragraphs AF-AL, wherein the absorbent fibers comprise cellulose ethyl sulphonate fibers.
AN. The dressing of any one of Paragraphs AF-AM, wherein the EPS-active agent comprises citric acid.
AO. The dressing of Paragraph AN, wherein the EPS-active agent comprises from 0.1% to 5% citric acid.
AP. The dressing of Paragraph AN or Paragraph AO, wherein the EPS-active agent comprises from 0.5% to 2.5% citric acid.
AQ. The dressing of any one of Paragraphs AF-AP, wherein the bioresorbable material comprises gelatin, collagen, oxidized regenerated cellulose (ORC), or a combination of any two or more thereof.
AR. The dressing of any one of Paragraphs AF-AQ, wherein the second layer is a film.
AS. The dressing of any one of Paragraphs AF-AR, further comprising a non-adherent layer, wherein the second layer is positioned between the first layer and the non-adherent layer.
AT. The dressing of Paragraph AS, wherein the non-adherent layer comprises ethyl methacrylate.
AU. The dressing of Paragraph AS or Paragraph AT, wherein the non-adherent layer is a film.
AV. A system for providing therapy to a tissue site, the system comprising a dressing of any one of Paragraphs AF-AU.
AW. A method for providing therapy to a tissue site, the method comprising positioning a dressing of any one of Paragraphs AF-AU adjacent to the tissue site.
AX. The method of Paragraph AW, further comprising providing negative pressure to the tissue site.
AY. A dressing comprising
   an absorbent, antimicrobial layer comprising absorbent fibers and antimicrobial fibers; and
   a bioresorbable layer comprising a bioresorbable material and an extracellular polymeric substance (EPS)-active agent.
AZ. The dressing of Paragraph AY, wherein the absorbent fibers comprise alginate fibers.
BA. The dressing of Paragraph AY or Paragraph AZ, wherein the absorbent fibers comprise carboxymethylcellulose.
BB. The dressing of any one of Paragraphs AY-BA, wherein the antimicrobial fibers comprise nylon fibers having an antimicrobial coating.
BC. The dressing of Paragraph BB, wherein the antimicrobial coating comprises silver.
BD. The dressing of any one of Paragraphs AY-BC, wherein the absorbent fibers comprise a cellulosic material.
BE. The dressing of any one of Paragraphs AY-BD, wherein the absorbent fibers comprise carboxymethylcellulose fibers and alginate fibers.
BF. The dressing of any one of Paragraphs AY-BE, wherein the absorbent fibers comprise cellulose ethyl sulphonate fibers.
BG. The dressing of any one of Paragraphs AY-BF, wherein the EPS-active agent comprises citric acid.
BH. The dressing of any one of Paragraphs AY-BG, wherein the EPS-active agent comprises citric acid, and wherein the bioresorbable layer comprises from 0.1% to 5% citric acid by weight to the bioresorbable layer.
BI. The dressing of any one of Paragraphs AY-BH, wherein the bioresorbable material comprises gelatin, collagen, oxidized regenerated cellulose (ORC), or a combination of any two or more thereof.
BJ. The dressing of any one of Paragraphs AY-BI, wherein the bioresorbable material comprises collagen and oxidized regenerated cellulose (ORC), where a weight ratio of collagen to ORC in the bioresorbable layer is about 60:40 to about 40:60.
BK. The dressing of any one of Paragraphs AY-BJ, wherein the bioresorbable layer is a film.
BL. The dressing of any one of Paragraphs AY-BK, wherein the dressing further comprises a non-adherent layer
BM. The dressing of Paragraph BL, wherein the bioresorbable layer is positioned between the absorbent, antimicrobial layer and the non-adherent layer.
BN. The dressing of Paragraph BL or Paragraph BM, wherein the non-adherent layer comprises ethyl methacrylate.
BO. The dressing of any one of Paragraphs BL-BN, wherein the non-adherent layer is a film.
BP. The dressing of any one of Paragraphs BL-BO, further comprising a cover positioned over the absorbent, antimicrobial layer, the bioresorbable layer, and the non-adherent layer, wherein the cover is configured to seal to tissue surrounding a tissue site.
BQ. The dressing of any one of Paragraphs AY-BP, wherein the absorbent fibers and antimicrobial fibers are nonwoven in the absorbent, antimicrobial layer.
BR. A method for providing therapy to a tissue site, the method comprising positioning a dressing of any one of Paragraphs AY-BQ adjacent to the tissue site.
BS. The method of Paragraph BR, further comprising providing negative pressure to the tissue site.

Other embodiments are set forth in the following claims, along with the full scope of equivalents to which such claims are entitled.

1. A dressing comprising
an absorbent, antimicrobial layer comprising absorbent fibers and antimicrobial fibers; and
a bioresorbable layer comprising a bioresorbable material and an extracellular polymeric substance (EPS)-active agent.

2. The dressing of claim 1, wherein the absorbent fibers comprise alginate fibers.

3. The dressing of claim 1, wherein the absorbent fibers comprise carboxymethylcellulose.

4. The dressing of claim 1, wherein the antimicrobial fibers comprise nylon fibers having an antimicrobial coating.

5. The dressing of claim 4, wherein the antimicrobial coating comprises silver.

6. The dressing of claim 1, wherein the absorbent fibers comprise a cellulosic material.

7. The dressing of claim 1, wherein the absorbent fibers comprise carboxymethylcellulose fibers and alginate fibers.

8. The dressing of claim 1, wherein the absorbent fibers comprise cellulose ethyl sulphonate fibers.

9. The dressing of claim 1, wherein the EPS-active agent comprises citric acid.

10. The dressing of claim 1, wherein the EPS-active agent comprises citric acid, and wherein the bioresorbable layer comprises from 0.1% to 5% citric acid by weight to the bioresorbable layer.

11. The dressing of claim 1, wherein the bioresorbable material comprises gelatin, collagen, oxidized regenerated cellulose (ORC), or a combination of any two or more thereof.

12. The dressing of claim 1, wherein the bioresorbable material comprises collagen and oxidized regenerated cellulose (ORC), where a weight ratio of collagen to ORC in the bioresorbable layer is about 60:40 to about 40:60.

13. (canceled)

14. The dressing of claim 1, wherein the dressing further comprises a non-adherent layer.

15. The dressing of claim 14, wherein the bioresorbable layer is positioned between the absorbent, antimicrobial layer and the non-adherent layer.

16. The dressing of claim 14 or claim 15, wherein the non-adherent layer comprises ethyl methacrylate.

17. The dressing of claim 14, wherein the non-adherent layer is a film.

18. The dressing of claim 15, further comprising a cover positioned over the absorbent, antimicrobial layer, the bioresorbable layer, and the non-adherent layer, wherein the cover is configured to seal to tissue surrounding a tissue site.

19. The dressing of claim 1, wherein the absorbent fibers and antimicrobial fibers are nonwoven in the absorbent, antimicrobial layer.

20. A method for providing therapy to a tissue site, the method comprising positioning a dressing of claim 1 adjacent to the tissue site.

21. The method of claim 20, further comprising providing negative pressure to the tissue site.
